(19) United States
(12) Patent Application Publication
(10) Pub. No.: US 2024/0226431 A1
(43) Pub. Date: Jul. 11, 2024

(54) Display Method and Apparatus of Medical Device, Electronic Device and Storage Medium

(71) Applicant: Medcaptain Medical Technology Co., Ltd., Shenzhen (CN)

(72) Inventors: Haoke HUANG, Shenzhen (CN); Jun DONG, Shenzhen (CN); Fulin LIU, Shenzhen (CN); Sheng SU, Shenzhen (CN); Ping LI, 518000 (CN)

(73) Assignee: Medcaptain Medical Technology Co., Ltd., Shenzhen (CN)

(21) Appl. No.: 18/530,197

(22) Filed: Dec. 5, 2023

(30) Foreign Application Priority Data
Jan. 5, 2023 (CN) 2023-10018678.1

(51) Int. Cl. A61M 5/168; G16H 30/20

(52) U.S. Cl. CPC A61M 5/16831; G16H 30/20; A61M 2205/13; A61M 2205/502

(57) ABSTRACT

The distance between a target user and a medical device is measured when it is determined that the target user is within a preset range of the medical device. The presence of the target user, who needs to be able to view a display of the medical device, is detected within a predetermined distance of the medical device. The distance between the target user and the medical device is then measured. Content, such as the amount of information, display font size, etc.., on a display of the medical device is automatically adjusted according to the measured distance between the target user and the medical device.

### REFERENCE TO RELATED APPLICATIONS

This application claims the priority of Chinese Patent Application No. 202310018678.1, entitled “Display Method and Apparatus of Medical Device, Electronic Device and Storage Medium” filed with the China National Intellectual Property Administration on Jan. 5, 2023, which is incorporated herein by reference in its entirety.

### TECHNICAL FIELD

This invention relates to the control of the display of a medical device, as well as to a corresponding electronic device and storage medium.

### BACKGROUND OF THE INVENTION

Medical devices such as infusion pumps and syringe pumps can automatically administer drugs to patients and monitor their treatment, and it can improve the efficiency and flexibility of clinical drug administration and reduce the workload of nursing.

However, when treating certain diseases, medical staff need to maintain a certain distance from the patients, for example, when a patient is located in an isolation ward for continuous infusion, and a doctor needs to pay attention to the patient's medication dosage and other information during ward rounds. Therefore, how to display the patient's medication and health status on medical devices in a timely manner, so that medical staff can view them efficiently, has become an urgent problem to be solved.

### SUMMARY OF THE INVENTION

Embodiments of the invention provide a display method and apparatus of a medical device, an electronic device and a storage medium to improve the display efficiency of the medical device.

In a first aspect, the invention provides a display method applied to a medical device, comprising:
determining a content to be displayed, generated by the medical device depending on the distance between the target and the medical device when it is determined that the target is within a preset range of the medical device; and
displaying the content to be displayed on a display interface of the medical device.

In a second aspect, the invention provides a display apparatus for use with a medical device, comprising:
a distance determination module configured to measure a distance between a target and the medical device when it is determined that the target is within a preset range of the medical device; wherein, the target is used to represent a user who is viewing the medical device;
a content determination module configured to determine a content to be displayed, generated by the medical device depending on the distance between the target and the medical device; and
a content display module configured to display the content to be displayed on a display interface of the medical device.

In a third aspect, the invention provides an electronic device, comprising a processor and a memory in communication with the processor; wherein
the memory stores computer-executable instructions; and
the processor is configured to execute the computer-executable instructions to implement the display method as described in the first aspect of the invention.

In a fourth aspect, the invention provides a computer-readable storage medium, comprising computer-executable instructions, which are executed by a processor to implement the display method as described in the first aspect.

In a fifth aspect, the invention provides a computer program product, comprising a computer program, which is executed by a processor to implement the display method as described in the first aspect of the invention.

The invention provides a display method and apparatus for a medical device, an electronic device and a storage medium, in which whether a target is located within a preset range of the medical device is determined. If yes, a distance between the target and the medical device is measured, and content to be displayed by the medical device is adjusted according to the distance. The content to be displayed is displayed on a screen of the medical device for viewing by the target. The invention solves the problems in the prior art that the user can only manually operate the medical device to display the content of the medical device, which causes low display efficiency and the like. It realizes the automatic adjustment of the display content and improves the display efficiency. By adjusting the content to be displayed according to the distance between the target and the medical device, the target can view the treatment of the patient from a distance, so that the target can maintain a certain distance from the patient, thereby ensuring the safety of the target, and improving the target's viewing efficiency of the content displayed on the medical device.

### BRIEF DESCRIPTION OF THE DRAWINGS

The accompanying drawings herein, which are incorporated into and form a part of the specification, illustrate embodiments consistent with the invention and are used in conjunction with the specification to explain the principles of the present application.

FIG. 1 shows a flowchart of a display method of a medical device in an embodiment of the invention.

FIG. 2a depicts a display interface in an embodiment of the invention.

FIG. 2b depicts the display interface in another embodiment of the invention.

FIG. 3 shows a flowchart of the display method of the medical device in an embodiment of the invention.

FIG. 4 shows a structural block diagram of a display apparatus of the medical device in an embodiment of the invention.

FIG. 5 shows a structural block diagram of the display apparatus of the medical device in another embodiment of the invention.

FIG. 6 shows a structural block diagram of an electronic device in an embodiment of the invention.

FIG. 7 shows a structural block diagram of an electronic device in another embodiment of the invention.

Embodiments of the invention are shown in the above drawings, which will be described in more detail later. These accompanying drawings and textual descriptions are not intended to limit the scope of the concept of the invention in any way, but rather to illustrate the concepts of invention for those skilled in the art by reference to particular embodiments.

### DETAILED DESCRIPTION

In order to make the purpose, technical solutions and advantages of the invention clearer, the embodiments of the invention will be further described in detail below in conjunction with the accompanying drawings.

It should be clear that the described embodiments are only some of the embodiments of the invention, not all of the embodiments. Based on the embodiments of the invention, all other embodiments obtained by persons of ordinary skill in the art without making creative efforts belong to the scope of protection of the invention.

When the following description refers to the accompanying drawings, the same numbers in different drawings indicate the same or similar elements unless otherwise indicated. The embodiments described in the following exemplary embodiments do not represent all embodiments consistent with the invention. Rather, they are only examples of apparatuses and methods consistent with some aspects of the invention as detailed in the appended claims.

In the description of the invention, it should be understood that the terms “first”, “second”, “third”, etc. are only used to distinguish similar objects, and are not necessarily used to describe a specific order or sequence, nor can they be read as indicating or implying relative importance. Those of ordinary skill in the art can understand the specific meanings of the above terms in this application according to specific situations. In addition, in the description of the invention, unless otherwise specified, “plurality” means two or more. “And/or” describes an associative relationship of associated objects, indicating that there may be three types of relationships, for example, A and/or B may indicate: A alone is present, A and B are present simultaneously, and B is present independently. The character “/” generally indicates that the contextual objects are an “or” relationship.

It should be noted that due to limited space, the specification does not exhaust all the optional embodiments. After reading the specification, those skilled in the art should be able to imagine that as long as the technical features do not contradict each other, then any combination of the technical features can constitute an optional embodiment. Each embodiment will be described in detail below.

Infusion pumps and syringe pumps, are instruments that can accurately control the flow rate of infusion to ensure that a drug can enter a patient's body safely at a uniform speed and in an accurate dose. Infusion pumps and syringe pumps can also improve the efficiency and flexibility of clinical drug delivery operations and reduce nursing workload.

When treating some infectious diseases, patients are usually located in the isolation ward, and the patients usually need continuous infusion. During ward rounds, the medical staff need to pay attention to the patient's medication situation. At such times, however, the medical staff typically need to be far away from the infusion pump, such that the medical staff cannot obtain clear information through the display of the pumps.

In addition to infusion pumps and syringe pumps, patients may also use other medical devices such as ventilators. During ward rounds, medical staff may also need to view the information displayed by various medical devices from a distance. Currently, medical staff are required to approach and manually operate the medical devices to display the contents of the medical devices. The display efficiency of the medical devices is low, and the medical staff are unable to view the information efficiently.

The invention provides a display method and apparatus for a medical device, an electronic device and a storage medium, aiming to solve the above technical problems in the prior art.

The technical solution of the invention and how the technical solution of the invention solves the above technical problems will be described in detail below with reference to specific embodiments. The following specific embodiments may be combined with each other, and the same or similar concepts or processes may not be repeated in some embodiments. Embodiments of the invention will be described below in conjunction with the accompanying drawings.

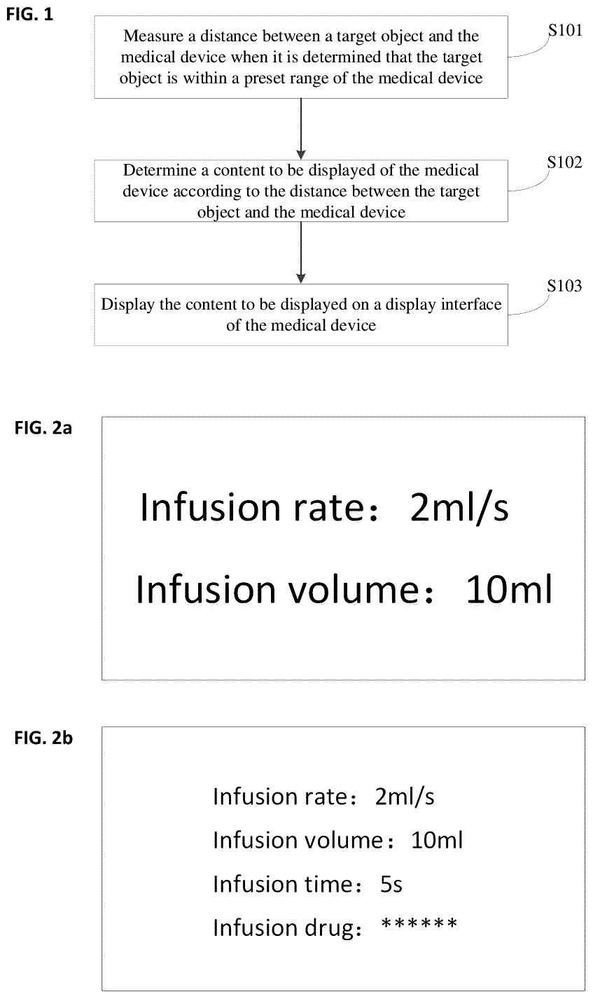

FIG. 1 is a flowchart of a display method for a medical device according to an embodiment of the invention, and the method is applied to a medical device and performed by a display apparatus of a medical device. As shown in FIG. 1, the method includes the following steps.

S101, if it is determined that a target is within a preset range of the medical device, a distance between the target and the medical device is measured. The target is used to represent a user who is viewing the medical device.

Exemplarily, the medical device may be a device used for treatment and care, for example, it may be an infusion pump, a syringe pump, or a ventilator, and the like. When a patient is using a medical device, a user is required to check the medical device to ensure the health and safety of the patient. For example, the user can be medical staff such as a doctor or a nurse. For some patients, the medical staff should not be too close and need to keep a certain distance from the patient to view the medical device. Therefore, it is necessary for the medical device to automatically display the treatment of the patient and the use of the medical device. For example, where the medical device is an infusion pump, the infusion pump can display infusion speed, infusion volume, and infusion medicine through its own display screen.

The medical device is provided with a target detection device, a distance detection device, and a processor. The target detection device detects whether or not a target is present within a preset range in real time or at regular intervals, and sends a detection result to the processor. The processor determines whether or not to measure the distance between the target and the medical device based on the detection result. If the detection result is that a target is located within the preset range, the distance detection device is controlled to measure the distance between the target and the medical device. The target detection device includes one or more of a millimeter wave radar, an infrared sensor, an ultrasonic sensor or an image acquisition device. The distance detection device includes one or more of a millimeter wave radar, an infrared sensor, an ultrasonic sensor or an image acquisition device.

In one example, the target detection device and the distance detection device may be the same device. For example, the medical device may be provided with a millimeter wave radar, and the millimeter wave radar may then used to determine both the presence of the target within the preset range and the distance between the target and the medical device. In another example, the target detection device and the distance detection device may be different devices, for example, the target detection device is an infrared sensor provided on the medical device, and the distance detection device is a millimeter wave radar provided on the medical device, such that the infrared sensor may be used for determining whether or not a target is present within the preset range, and the millimeter wave radar is used for measuring the distance between the target and the medical device.

A preset range of the medical device is set in advance, and the medical device determines whether there is a target present within the preset range, and if there is, the medical device may measure the distance between the target and the medical device. For example, the preset range may be a range centered on the medical device with a preset distance as a radius. The target may be a user who needs to view the medical device, for example, it may be a medical staff member responsible for checking a ward. The medical device may determine in real time whether there is a medical staff member within the preset range, that is, determine whether there is a medical staff member near the medical device. If so, the medical device may determine that the medical staff member is a target, that is, there is a target within the preset range, and then measure the distance between the medical staff member and the medical device. For example, a camera or other equipment may be arranged on the medical device, and images within the preset range may be acquired by the camera, and a preset target recognition algorithm may be used to identify whether there is a target within the preset range. Based on the position of the target in the image, a distance between the target and the medical device can be determined. In this embodiment, the preset target recognition algorithm is not specifically limited. For example, the target recognition algorithm may be a face recognition algorithm.

The target may be any user or a pre-registered member of the medical staff. For example, as long as a user is within the preset range of the medical device, the user can be considered as a target, and the distance between the user and the medical device is measured. Alternatively, the distance between the medical staff member and the medical device may be measured only if a pre-registered identified medical staff member is within the preset range. For example, when someone is within the preset range, it is determined whether the user within the preset range is a pre-registered medical staff member by face recognition, and if so, the user is treated as a target, and the distance between the user and the medical device is measured, and if not, there is no need to measure the distance between the user and the medical device.

S102, according to the distance between the target and the medical device, the content to be displayed for the medical device is determined.

Exemplarily, the distance between the target and the medical device may be determined as a target distance, and after the target distance is determined, the content to be displayed for the medical device may be determined according to the target distance. The correlation between different distances and contents to be displayed may be preset, and after the target distance is determined, the content to be displayed associated with the target distance may be determined. For example, when the distance between the target and the medical device is 1 meter, the associated contents to be displayed might be the infusion speed, the infusion time, the infusion volume, and the infused drug; when the distance between the target and the medical device is 5 meters, the associated contents to be displayed might be only the infusion speed and the infusion volume.

The content to be displayed may include displayed data such as the treatment condition of the patient or the use condition of the medical device, and different contents to be displayed may correspond to different display modes. For example, the display mode may include a font size and a font color of the displayed data, and the like. The further the distance between the target and the medical device, the larger the font size may be and the brighter the font color may be. For example, when the distance between the target and the medical device is 1 meter, the font color is white; when the distance between the target and the medical device is 5 meters, the font color is bright yellow.

A medical device such as an infusion pump or other medical device may include a display, and the content to be displayed may be adjusted according to the distance between the target and the medical device. When the target is far away from the medical device, the font of the content can be enlarged. For example, if the information to be displayed might be only the name of the drug and the infusion rate, which allows the medical staff to observe the important content more clearly. When as distance between the target and the medical device becomes smaller, more content can be added, such as the amount of the infusion. When the target is standing in front of the medical device, for example, the target distance is within 1 meter, then the medical device might display all the information required by the target.

In this embodiment, the content to be displayed for the medical device is determined according to the distance between the target and the medical device, comprising: according to the distance between the target and the medical device and a preset correlation between the distance and a display mode, determining the content to be displayed for the medical device, as well as a rotation mode, font size and font color of the content to be displayed.

Specifically, a correlation between different distances and display modes is preset, for example, a correlation between different distance ranges and the display modes may be preset. After obtaining the distance between the target and the medical device, a distance range within which the target is located is determined, and the corresponding display mode is determined according to the preset correlation. For example, when the distance between the target and the medical device is between 1 meter and 2 meters, the corresponding display mode may be to display real-time data, such as the infusion speed and the infusion volume, on the display screen in a size of Song font No. 4 and a white font color.

The display mode may include specific content to be displayed, as well as a rotation mode, a font size, a font color of the content to be displayed, and the like. The rotation mode means that when the display screen of the medical device is unable to display all of the contents to be displayed at once, the contents to be displayed may alternately appear on the display screen according to a preset rotation mode. For example, if the contents to be displayed include an infusion rate and an infusion volume, the rotation mode might be that the infusion rate and the infusion volume are alternately scrolled on the display. Alternatively, a rotation character bar may also be preset, and the rotation character bar may be a separator between different contents to be displayed. For example, the rotation character bar may be “|”, and if the contents to be displayed are the infusion rate and the infusion volume, then the infusion rate and the infusion volume may be separated by “|”.

The distance between the target and the medical device can be determined in real time, and the display mode can be changed in real time according to the distance between the target and the medical device. For example, a font size of the content to be displayed may become smaller as the target approaches and larger as the target moves away.

The beneficial effect of this setting is that the content to be displayed and the display mode are adjusted in real time according to the distance between the target and the medical device, so that the medical device can carry out the display in a timely manner and display the content required by the target, and the display efficiency and accuracy of the medical device are improved. The target can clearly view the medical device from a distance and improve the viewing experience of the target.

In this embodiment, the medical device includes a first device and a second device; determining the content to be displayed by the medical device based on a distance between the target and the medical device, comprising: if the distance between the target and the first device is obtained, determining the content to be displayed on the second device based on the distance between the target and the first device.

Patients may sometimes use multiple medical devices at the same time, and medical staff need to check the use of multiple medical devices during ward rounds. For example, if a patient is using an infusion pump and a ventilator at the same time, the medical staff needs to maintain a certain distance from the patient to check the infusion rate displayed by the infusion pump and the respiratory rate displayed by the ventilator. As another example, if a patient is using more than one infusion pump at the same time, the medical staff needs to obtain data, such as the infusion rate and infusion drug, displayed by each infusion pump.

If a patient is using two medical devices at the same time, one of the medical devices is the first device and the other medical device is the second device. If the patient is using three or more medical devices at the same time, one of the medical devices is the first device, and the other medical devices are the second device. The first device is the medical device that first determines the presence of the target within its preset range. For example, if the medical staff is approaching the patient, and the medical staff is oriented directly to the infusion pump, and the infusion pump is the first to recognize the target, then the infusion pump may be the first device, and the other medical devices are the second devices. The first device and the second device of the plurality of devices may also be preset.

The first device determines a distance between the target and itself after determining that the target is present within a preset range. Based on the distance between the target and the first device, content to be displayed by the first device is determined. Both the second device and the first device serve the same patient, and the first device and the second device are located in the same ward. After obtaining the distance between the target and the first device, the distance between the target and the first device can be used as the distance between the target and the second device, so as to determine the content to be displayed on the second device. That is, the content to be displayed for the second device is determined based on the distance between the target and the first device.

The beneficial effect of this setting is that according to the distance obtained by one medical device, the content to be displayed of all other medical devices can be dynamically adjusted. When the font of the content to be displayed on the first device becomes larger, the font of the content to be displayed on the second device may also become larger. This setting can simplify the process of recognition of the target by the second device, improving the display efficiency of the medical device, and thus improving the viewing efficiency of the user.

Multiple medical devices can also individually determine whether a target is present within a preset range, and determine the distance between the target and each medical device. Distances determined by each medical device can be verified, for example, each medical device may obtain the distances determined by the other medical devices, and determine whether the difference between the distances is within a preset difference range. If a medical device determines that among the distance differences between itself and the other medical devices, there is a distance difference that lies within the preset difference range, then the medical device may determine its own content to be displayed according to the distance determined by itself. If a medical device determines that the distance differences between itself and other medical devices are not within the preset difference range, then it considers that the distance obtained by itself is erroneous. In this case, the identification of the target and the determination of the distance can be repeated, or the distance obtained by other medical devices can be used as the distance between the medical device and the target. This enables the adjustment of the distance and improves the accuracy of the display. For example, assume there are three medical devices, device one, device two, and device three. Each medical device determines distance differences between the distance obtained by itself and the distances obtained by the other two medical devices. Assume further that the distance difference between device one and device two is within the preset difference range; the distance difference between device two and device three is not within the preset difference range; and the distance difference between device one and device three is not within the preset difference range. It can be assumed that the distance obtained by device three is erroneous, and device three can re-determine the distance, or use the distance obtained by device one or device two as the distance for device three, to improve the accuracy of the content display of device three.

S103, the content to be displayed is displayed on a display interface of the medical device.

For example, a display screen may be arranged on the medical device for displaying a display interface from which the content to be displayed is displayed. After the content to be displayed is determined, the data of the content to be displayed can be displayed on the display interface of the medical device according to a display manner. For example, the information may be displayed according to a preset font size and font color. As the target moves, the content to be displayed on the display interface may change. For example, the display font may become larger as the target is away from the medical device.

FIG. 2(a) shows a schematic diagram of a display interface in the embodiment when the distance between the target and the medical device is at a first distance, and FIG. 2(b) shows a schematic diagram of a display interface in the embodiment when the distance between the target and the medical device is at a second distance. The first distance is greater than the second distance, and the medical device in this example is an infusion pump. The font of the content to be displayed in FIG. 2(a) is larger and less data is displayed; and the font of the content to be displayed in FIG. 2(b) is smaller and more data is displayed.

This embodiment of the invention provides a display method applied to the medical device, by determining whether a target is present within a preset range of the medical device, and if so, measuring a distance between the target and the medical device. Content to be displayed for the medical device is adjusted according to the distance. The content to be displayed is displayed on a screen of the medical device for viewing by the target. The invention solves the problems in the prior art that the user can only manually operate the medical device to display the content of the medical device, which causes low display efficiency and the like. It realizes automatic adjustment of the display content and improves display efficiency. Adjusting the display content according to the distance between the target and the medical device makes it possible for the target to view the treatment of the patient from a distance, so that the target can maintain a certain distance from the patient, ensure the safety of the target, and improve the target's viewing efficiency of the content displayed on the medical device.

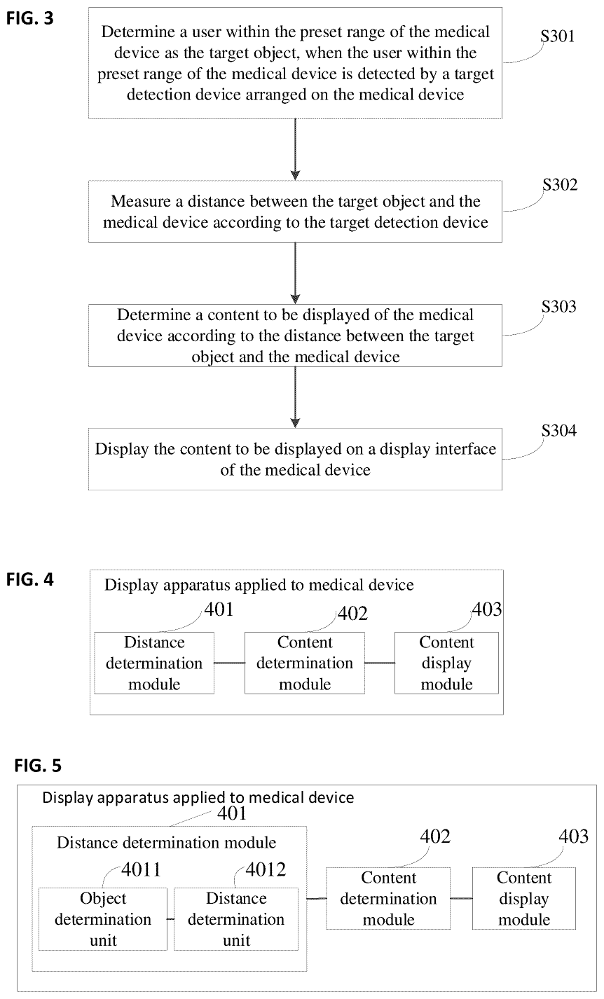

FIG. 3 shows a flow diagram of a display method of a medical device in an embodiment of the invention, which is an optional embodiment based on the above embodiment.

In this embodiment, if it is determined that a target is within a preset range of the medical device, then the system determines a distance between the target and the medical device, which may be refined as follows: identifying a user within the preset range of the medical device as the target, if the user within the preset range of the medical device is detected according to a target detection device arranged on the medical device. The target detection device includes one or more of these devices, which may be a millimeter wave radar, an infrared sensor, an ultrasonic sensor, or an image acquisition device. Based on the target detection device, a distance between the target and the medical device is measured.

As shown in FIG. 3, the method includes the following steps.

S301, based on the target detection device arranged on the medical device, if it is determined that a user is present within a preset range of the medical device, the user will be considered as a target; and the target detection device may include one or more of these devices, which may be a millimeter wave radar, an infrared sensor, an ultrasonic sensor or an image acquisition device, and the image acquisition device may be a camera and the like.

As one example, one or more target detection devices may be arranged on the medical device, for example, the target detection device may include one or more of these devices, which may be a millimeter wave radar, an infrared sensor, an ultrasonic sensor or an image acquisition device, and the image acquisition device may be a camera and the like.

The medical device may detect whether a target is present within the preset range in real time or at regular intervals by its own target detection device. For example, the target detection device determines whether a user is present within the preset range of the medical device, and if so, the user, which is located within the preset range, is determined to be the target. If the target detection device is a millimeter wave radar, based on the millimeter wave radar, millimeter-wave transmission and reception can be performed within the preset range to determine whether a user is present. If the target detection device is an infrared sensor, based on the infrared sensor, detecting whether infrared heat corresponding to a human body is present within the preset range, thereby, it can be determined whether a user is present within the preset range. If the target detection device is an ultrasound sensor, based on the ultrasonic sensor, then ultrasonic waves are sent and received within the preset range to determine whether a user is present within the preset range. If the target detection device is an image acquisition device, based on the image acquisition device, an image or a video within the preset range can be acquired, and by means of a preset target recognition algorithm, whether a user is present in the image or the video can be determined.

The identification condition for the target may be set in advance, and after determining that a user is present within a preset range of the medical device according to the target detection device arranged on the medical device, identifying information for the user is obtained, and determining whether the identifying information for the user satisfies the preset identification conditions, and if so, determining that the user is the target; if not, determining that the user is not the target. The identification information may be information identifying whether the user is the target, for example, it may be identity information of the user, or it may be information such as how long the user is located within the preset range, that is the user's “dwell time”, and the identity information may be information such as a human face. For example, if the preset identification condition is that the medical device will display content only when a specific medical staff member enters the preset range, the identity information of the user in the preset range can be obtained to determine whether the user is a predetermined specific medical staff, and thus whether the user is the target.

In this embodiment, if it is detected that a user is present within a preset range of the medical device, based on the target detection device arranged on the medical device, the user may be considered as a target. The method of operation then comprises: if it is detected that a user is present within a preset range of the medical device, based on the target detection device arranged on the medical device, obtaining a dwell time of the user within the preset range; and determining that the user is a target if the dwell time is more than a preset time threshold.

Specifically, if a user, such as a medical staff member, just passes by the ward, there is no need to change the display interface of the medical device at this time. The user needs to stay within a preset range of the medical device for a certain period of time to trigger the medical device to carry out the determination of the target, and the time can be set according to the actual needs.

Whether a user is present within the preset range is detected by the target detection device, and if so, the dwell time of the user within the preset range is determined. For example, the presence of a user within the preset range may be detected by the video captured by the camera. A point in time at which the user appears in the video and a point in time at which the user disappears from the video are determined to obtain the dwell time of the user. A time threshold is preset, the dwell time is compared with the preset time threshold, and if the dwell time exceeds the preset time threshold, the user is determined to be the target; and if the dwell time does not exceed the preset time threshold, the user is determined not to be the target. If there a plurality of users are located within the preset range whose dwell times all exceed the preset time threshold, the distance between each user and the medical device is detected and the user closest to the medical device is identified as the target.

The beneficial effect of such a setting is that by performing a comparison of the dwell time, it avoids mistaking a passing user for the target, reduces the occurrence of a misrecognition situation, improves the detection accuracy of the target, avoids frequent content display by the medical device, and improves the display efficiency and precision of the medical device.

In this embodiment, if a user is detected within a preset range of the medical device, based on the target detection device arranged on the medical device, the user will be considered as a target. The method of operation then comprises: if a user is detected within a preset range of the medical device, based on the target detection device arranged on the medical device, a distance change between the user and the medical device is determined, and if it is determined that the distance change between the user and the medical device satisfies a preset change state, the user is determined to be a target.

Specifically, based on a target detection device arranged on the medical device, it is determined whether a user is present within a preset range of the medical device, and if so, a movement trajectory of the user is detected to obtain a distance change between the user and the medical device. The distance change means that the user is getting closer to or farther away from the medical device. For example, if the target detection device is an infrared sensor, the distance change between the user and the medical device can be determined by the size of the infrared imaging surface. If the infrared imaging surface is becoming larger, the distance change is determined as getting closer; if the infrared imaging surface is becoming smaller, the distance change is determined as getting farther away. As another example, if the target detection device is a millimeter wave radar, the distance change between the user and the medical device can be determined by the size of the reflected surface of the millimeter wave. If the reflective surface becomes larger, the distance change is determined as getting closer; if the reflective surface becomes smaller, the distance change is determined as getting farther away. The distance change between the user and the medical device can also be determined by, for example, image recognition by a camera.

A change condition is preset, and the preset change condition may be that the distance change is getting closer. If it is determined that the distance change between the user and the medical device satisfies the preset change condition, that is, it is determined whether the distance change between the user and the medical device is getting closer, then the user is determined to be the target; if the distance change between the user and the medical device is getting farther away, it is determined that the user is not the target.

If there are a plurality of users within the preset range, the user who is approaching the medical device may be determined as the target based on the distance changes corresponding to the plurality of users. If there is a plurality of users who are approaching the medical device, a distance between each of the users and the medical device can be determined, and the closest of these users to the medical device can be identified as the target.

The beneficial effect of such a setting is that a target detection device on the medical device, such as a sensor, can detect a change in the relative position of the user within a preset range, and when the user is sensed as being approaching, the target can be determined in advance, which facilitates the timely adjustment of the content to be displayed, and improves the efficiency and precision of the display.

In this embodiment, assume that at least two users are present within a preset range of the medical device; if it is determined that a user is present within the preset range of the medical device according to the target detection device arranged on the medical device, the system determines that the user is a target. This method then comprises: measuring an orientation angle of each user relative to the medical device; and determining that the user as a target if the orientation angle of the user satisfies a preset angle condition.

Specifically, the target detection device on the medical device may measure an orientation angle of the user relative to the medical device, that is, it may determine whether the user is facing the medical device. For example, an infrared imaging surface size may be determined by an infrared sensor, a size threshold of the infrared imaging surface may be predetermined, and if the size of the infrared imaging surface exceeds the preset size threshold of the infrared imaging surface, it may be assumed that the user is facing the medical device; or a reflective surface size may be determined by millimeter wave radar, a size threshold of the reflective surface may be preset, and if the size of the reflective surface exceeds the preset size threshold of the reflective surface, it may be assumed that the user is facing the medical device. It may also be recognized by the camera whether the user is facing the medical device. If the user is facing the medical device, the user's orientation angle may be determined to be 180 degrees.

If an angle condition is preset, for example, the angle condition may be an range of the user's orientation angle toward the medical device, and if the user's orientation angle toward the medical device is within a preset angular range, the user may be considered to be facing the medical device, such that the user is considered to be the target.

A plurality of users may be located within a preset range of the medical device. If a plurality of users are present, an orientation angle of each user toward the medical device may be detected. Whether each orientation angle is within the preset angle range is determined, that is, it is determined whether each orientation angle satisfies the preset angle condition. If so, then the user corresponding to the orientation angle is considered as the target. An angle threshold may be preset, and if a plurality of orientation angles are detected within the preset angle range, a difference between the orientation angles located within the angle range and the angle threshold is determined, and the user corresponding to the orientation angle with the smallest difference is identified as the target, that is, the user corresponding to the orientation angle that is closest to the angle threshold is identified as the target. If a plurality of orientation angles are located within the predetermined angle range, a distance between the user whose orientation angle located within the angle range and the medical device may also be measured, and the user with the smallest distance is considered as the target.

The beneficial effect of such a setting is that when there is a plurality of users are within a preset range, the optimal user facing the medical device can be determined by means of the size of the infrared imaging surface, the size of the reflective surface, the camera identification and the like, so as to carry out a content display for the user, and to improve the precision and efficiency of the display.

In this embodiment, one or more users may be located within a preset range of the medical device, and if it is determined that only one user is present within the preset range of the medical device based on the target detection device arranged on the medical device, an orientation angle of the user toward the medical device may be measured; if the orientation angle of the user satisfies the preset angle condition, the user is considered to be the target; and if the orientation angle of the user does not satisfy the preset angle condition, the user is not the target. That is, for each user within the preset range, the user's orientation angle can be measured, and even if only one user is present within the preset range, the target may not be present, realizing that the medical device can accurately determine the target, display the content for the target, and effectively improve the display accuracy.

In this embodiment, at least two users are present within a preset range of the medical device; if it is determined that a user is present within the preset range of the medical device based on the target detection device arranged on the medical device, the user located within the preset range of the medical device is considered to be the target. The method in this embodiment comprises: based on the pre-arranged image acquisition device, acquiring a face image of each of the users; and determining the user to be a target if the face image of the user is stored in a preset face image library.

Specifically, the medical device may be arranged with an image acquisition device, such as a camera, and the medical device may acquire a facial image of a user within a preset range by means of the image acquisition device. A human facial image library is pre-stored in the medical device, and facial information of the preset user is stored in the human facial image library, for example, facial information of a medical staff member is stored in the human facial image library. After obtaining the facial image of the user, it is determined whether the facial image of the user is stored in the facial image library, and if so, it is determined that the user has the permission to view the medical device, and that the user is the target; if not, it is determined that the user does not have the permission to view the medical device, and that the user is not the target.

A plurality of users may be located within a preset range of the medical device, and if a plurality of users are present, a facial image of each of the users may be acquired. The system then determines whether or not each of the facial images is located in the facial image library, and the user corresponding to the facial image located in the facial image library is considered as a target. If there are a plurality of users whose facial images are all located in the face image library, the plurality of users located in the facial image library are determined as candidate targets. The distance between each candidate target and the medical device is then determined, and the closest candidate target is identified as the target.

The beneficial effect of such a setting is that by performing facial recognition, it can be ensured that only professionals can view the medical device, avoiding misrecognition and information leakage, and improving the efficiency and precision of content display.

In this embodiment, one or more users may be located within a preset range of the medical device, and if it is detected that only one user is present within the preset range of the medical device based on the target detection device arranged on the medical device, a facial image of the user may be acquired based on the pre-arranged image acquisition device; if the facial image of the user is stored in the preset facial image library, the user is determined to be the target. If the facial image of the user is not in the preset facial image library, it is determined that the user is not the target. That is, for each user within the preset range, the facial image of the user can be determined without limit of the number of people. Even if only one user is present within the preset range, the target may not be present, realizing that the medical device can accurately determine the target and display the content for the target, further improving the display accuracy.

A medical device may be pre-arranged with a plurality of target detection devices, for example, the medical device may have both an infrared sensor and a camera. A multi-sensor fusion can be used to determine whether a user within a preset range is a target.

In this embodiment, the target detection device on the medical device may include a first detection device and a second detection device. Based on the target detection device arranged on the medical device, if it is determined that a user is present within a preset range of the medical device, the user who is present within the preset range of the medical device is determined to be the target. The method according to this embodiment comprises: based on the first detection device arranged on the medical device, determining whether a user is present within a predetermined range of the medical device; if so, determining whether the user satisfies a preset confirmation condition based on the second detection device arranged on the medical device; and if so, the user is considered as a target.

Specifically, the first detection device may be a target detection device for detecting a distance between the user and the medical device, and the second detection device may be a target detection device other than the first detection device. By means of the first detection device, the distance between the user and the medical device may be detected to determine whether the user is within a preset range of the medical device. If not, no further target determination is required for the user; if so, by the second detection device, it is determined whether the user satisfies the preset confirmation condition. If it is satisfied, it is determined that the user is the target; if it is not satisfied, it is determined that the user is not the target. The confirmation condition may be determined according to the second detection device. For example, if the second detection device is a camera, the confirmation condition may be that the facial image of the user is located in a pre-stored facial image library; or, if the second detection device is an infrared sensor, the confirmation condition may be that the user's facing angle is within the preset angle range. That is, the distance between the user and the medical device may be judged first, and the recognition of the facial image may be initiated after the distance satisfies the condition of the preset range; or, the distance between the user and the medical device may be judged first, and the orientation angle of the user may be judged after the distance satisfies the condition of the preset range.

Three or more target detection devices may also be arranged on the medical device, whereby the user is determined to be the target only after the user satisfies the confirmation conditions of all the target detection devices. For example, a distance between the user and the medical device may first be determined, and if the distance is within the preset range, then a facial image of the user is acquired, and if the facial image of the user is stored in a preset facial image library, a facing angle of the user is acquired. If the user's facing angle is within the preset angle range, the user is determined to be the target. The present embodiment does not specifically limit the order of evaluation of each condition.

The beneficial effect of such a setting is that, by means of multi-sensor fusion, precise determination of the target can be realized, avoiding identifying a passing user or a user without authority as the target, improving the precision of determination of the target, and thus improving the display precision of the medical device.

S302, based on the target detection device, the distance between the target and the medical device is determined.

As one example, after determining the target, a distance between the target and the medical device is measured based on the target detection device. For example, a coordinate position of the target in an image may be determined by a camera, and based on the coordinate position, the distance between the target and the medical device may be calculated. Different target detection devices may be used to determine the distance in different ways. The invention is not limited to any specific way of determining the distance.

S303, based on the distance between the target and the medical device, the content to be displayed for the medical device is determined.

As one example, this step can be referred to the above step S102 and not be repeated.

S304, the content to be displayed is displayed on a display interface of the medical device.

As one example, this step may be referred to the above step S103, and not be repeated.

The invention provides a display method for a medical device, by determining whether a target is present within a predetermined range of the medical device, and if so, determining a distance between the target and the medical device. Content to be displayed by the medical device is adjusted according to the distance. The content to be displayed is displayed on a screen of the medical device for viewing by the target. The invention solves the problems in the prior art that the user can only manually operate the medical device to display the content of the medical device, which causes low display efficiency and the like. It provides automatic adjustment of the display content and improves display efficiency. Adjusting the display content according to the distance between the target and the medical device makes it possible for the target to view the treatment of the patient from a distance, so that the target can maintain a certain distance from the patient, ensure the safety of the target, and improve the target's viewing efficiency of the content displayed on the medical device.

FIG. 4 shows a structural block diagram of a display apparatus of a medical device in an embodiment of the invention, which is applied to a medical device. For ease of illustration, only the portions relevant to the embodiments of the invention are shown. As shown in FIG. 4, the apparatus includes a distance determination module 401, a content determination module 402, and a content display module 403.

The distance determination module 401 is configured to determine a distance between the target and the medical device if it is determined that, within a preset range of the medical device, there is a target. “Target” denotes a user who is viewing the medical device.

The content determination module 402 is configured to determine content to be displayed for the medical device based on the distance between the target and the medical device.

The content display module 403 is configured to display the content to be displayed on a display interface of the medical device.

FIG. 5 shows a structural block diagram of a display apparatus of a medical device in an embodiment of the invention, and on the basis of the embodiment shown in FIG. 4, as shown in FIG. 5, the distance determining module 401 includes an object determining unit 4011 and a distance determining unit 4012.

The object determining unit 4011 is configured to determine a user as the target if the presence of the user is detected within a preset range of the medical device according to a target detection device arranged on the medical device; and the target detection device may include one or more of these devices, which may be a millimeter wave radar, an infrared sensor, an ultrasonic sensor or an image acquisition device.

The distance determining unit 4012 is configured to determine a distance between the target and the medical device, based on the target detection device.

In one example, the object determination unit 4011, is specifically configured to:
determine the dwell time of the user within the preset range, if it is determined that a user is present within the preset range of the medical device based on the object detection device arranged on the medical device; and
determine the user to be the target if the dwell time exceeds a preset time threshold.

In one example, the object determination unit 4011, is specifically configured to:
determine a distance change between the user and the medical device, if it is determined that a user is present within the preset range of the medical device based on the object detection device arranged on the medical device; and
determine the user to be the target if the distance change between the user and the medical device satisfies a preset change state.

In one example, at least two users are located within a preset range of the medical device.

An object determination unit 4011, is specifically configured to:
determine an orientation angle of each user towards the medical device; and
determine the user as the target if the orientation angle of the user satisfies a preset angle condition.

In one example, at least two users are located within a preset range of the medical device.

An object determining unit 4011, is specifically configured to:
acquire a face image of each user based on a pre-arranged image acquisition device; and
determine the user as the target if the face image of the user is stored in a preset face image library.

In one example, the content determination module 402, is specifically configured to:
determine content to be displayed for the medical device based on a preset correlation between the distance and the display method, and determine a rotation mode, a font size, and a font color for the content to be displayed, based on a distance between the target and the medical device.

In one example, the medical device includes a first device and a second device.

A content determination module 402, is specifically configured to:
determine content to be displayed for the second device based on the distance between the target and the first device, if a distance between the target and the first device is determined.

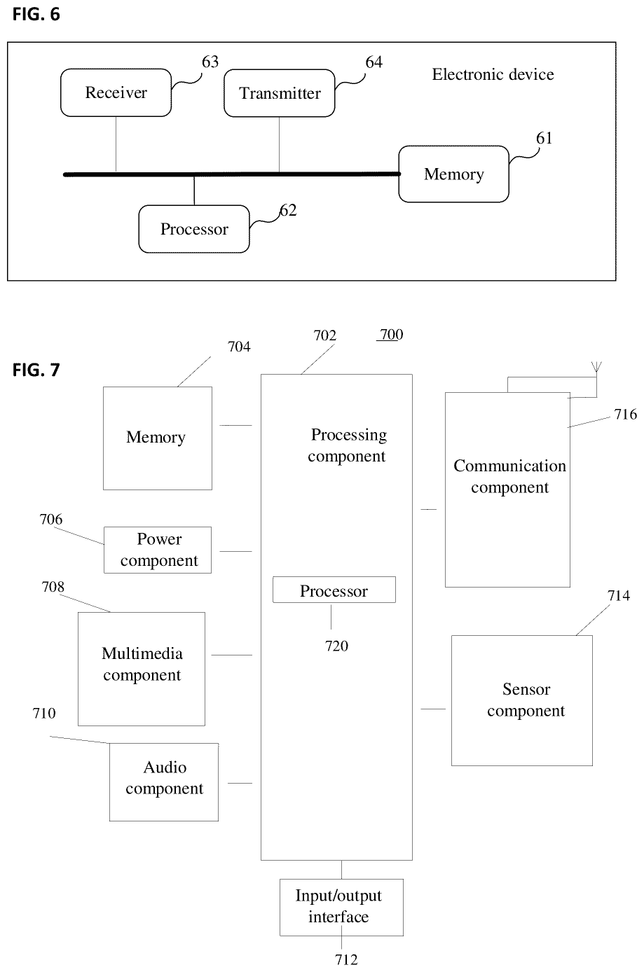

FIG. 6 shows a structure block diagram of an electronic device in an embodiment of the invention. As shown in FIG. 6, the electronic device includes: a memory 61, a processor 62, in which the memory 61 is provided for storing executable instructions of the processor 62.

The processor 62 is configured to perform a method as provided in the embodiments described above.

The electronic device also includes a receiver 63 and a transmitter 64. The receiver 63 is used to receive commands and data sent by other devices, and the transmitter 64 is used to send commands and data to an external device.

The processor 62 is configured to perform the method as provided in the embodiments described above. The method specifically includes:
determining content to be displayed of the medical device according to a distance between a target and the medical device when it is determined that the target is within a preset range of the medical device; and
displaying the content to be displayed on a display interface of the medical device.

In one example, the step of when it is determined that the target is within the preset range of the medical device includes:
determining a user within the preset range of the medical device as the target, when the user within the preset range of the medical device is detected by a target detection device arranged on the medical device. The target detection device comprises one or more of a millimeter wave radar, an infrared sensor, an ultrasonic sensor or an image acquisition device.

In one example, the step of determining the user within the preset range of the medical device as the target, when the user within the preset range of the medical device is detected by the target detection device arranged on the medical device, includes:
obtaining a dwell time of the user within the preset range, when the user within the preset range of the medical device is detected by the target detection device arranged on the medical device;
determining the user as the target when the dwell time is more than a preset time threshold.

In one example, the step of determining the user within the preset range of the medical device as the target, when the user within the preset range of the medical device is detected by the target detection device arranged on the medical device, includes:
obtaining a distance change between the user and the medical device when the user within the preset range of the medical device is detected by the target detection device arranged on the medical device;
determining the user as the target when the distance change between the user and the medical device satisfies a preset change state.

In one example, the step of determining the user within the preset range of the medical device as the target when the user within the preset range of the medical device is detected by the target detection device arranged on the medical device, includes:
measuring an orientation angle of each user to the medical device;
determining the user as the target when the orientation angle of the user satisfies a preset angle condition.

In one example, the step of determining the user within the preset range of the medical device as the target when the user within the preset range of the medical device is detected by the target detection device arranged on the medical device, includes:
acquiring a facial image of each user according to a pre-arranged image acquisition device;
determining the user as the target when the facial image of the user is stored in a preset face image library.

In one example, the step of determining the content to be displayed of the medical device according to the distance between the target and the medical device, includes:
according to the distance between the target and the medical device and a preset correlation between the distance and a display mode, determining the content to be displayed and/or a display mode of the medical device.

In one example, the medical device comprises a first device and a second device and the step of determining the content to be displayed of the medical device according to the distance between the target and the medical device, includes:
determining the content to be displayed of the first device or/and the second device according to a distance between the target and the first device.

FIG. 7 is a structural block diagram of an electronic device illustrated according to an exemplary embodiment, which may be a cell phone, a computer, a digital broadcasting terminal, a message transceiver device, a game console, a tablet device, a medical device, a fitness device, a personal digital assistant, and the like.

The device 700 may include one or more of the following components: a processing component 702, a memory 704, a power component 706, a multimedia component 708, an audio component 710, an input/output (I/O) interface 712, a sensor component 714, and a communication component 716.

The processing component 702 generally controls the overall operation of the device 700, such as operations associated with displays, telephone calls, data communications, camera operations, and recording operations. The processing component 702 may include one or more processors 720 to execute instructions to accomplish all or some of the steps of the method described above. In addition, the processing component 702 may include one or more modules to facilitate interaction between the processing component 702 and other components. For example, the processing component 702 may include a multimedia module to facilitate interaction between the multimedia component 708 and the processing component 702.

The memory 704 is configured to store various types of data to support operation at the device 700. Examples of such data include instructions for any application or method operating on the device 700, contact data, phone book data, messages, pictures, videos, etc. The memory 704 may be implemented by any type of volatile or non-volatile storage device or a combination thereof, such as static random access memory (SRAM), electrically erasable programmable read-only memory (EEPROM), erasable programmable read-only memory (EPROM), programmable read-only memory (PROM), read-only memory (ROM), magnetic memory, a flash memory, a Disks or CD-ROMs.

The power supply assembly 706 provides power to various components of the device 700. The power supply assembly 706 may include a power management system, one or more power sources, and other components associated with generating, managing, and distributing power for the device 700.

The multimedia component 708 includes a screen providing an output interface between the device 700 and the user. In some embodiments, the screen may include a liquid crystal display (LCD) and a touch panel (TP). If the screen includes a touch panel, the screen may be implemented as a touch screen to receive input signals from the user. The touch panel includes one or more touch sensors to sense touches, swipes and gestures on the touch panel. The touch sensors may not only sense the boundaries of a touch or swipe action, but also detect the duration and pressure associated with the touch or swipe operation. In some embodiments, the multimedia component 708 includes a front camera and/or a rear camera. The front camera and/or the rear camera may receive external multimedia data when the device 700 is in an operational mode, such as a shooting mode or a video mode. Each front camera and rear camera may be a fixed optical lens system or have a focal length and optical zoom capability.

The audio component 710 is configured to output and/or input audio signals. For example, the audio component 710 includes a microphone (MIC) that is configured to receive external audio signals when the device 700 is in an operational mode, such as a call mode, a recording mode, and a voice recognition mode. The received audio signals may be further stored in memory 704 or sent via communication component 716. In some embodiments, the audio component 710 further includes a speaker for outputting audio signals.

The I/O interface 712 provides an interface between the processing component 702 and a peripheral interface module, the peripheral interface module may be a keypad, a click wheel, a button, and the like. These buttons may include, but are not limited to: a home button, a volume button, a start button, and a lock button.

The sensor assembly 714 includes one or more sensors for providing an assessment of various aspects of the state of the device 700. For example, the sensor assembly 714 may detect an open/closed state of the device 700, relative positioning of components, such as the components being the display and keypad of the device 700, the sensor assembly 714 may also detect a change in the position of the device 700 or a component of the device 700, the presence or absence of user contact with the device 700, the orientation or acceleration/deceleration of the device 700, and a change in temperature of the device 700. The sensor assembly 714 may include a proximity sensor configured to detect the presence of a nearby object in the absence of any physical contact. The sensor assembly 714 may also include a light sensor, such as a CMOS or CCD image sensor, for use in imaging applications. In some embodiments, the sensor assembly 714 may also include an acceleration sensor, a gyroscope sensor, a magnetic sensor, a pressure sensor, or a temperature sensor.

The communication component 716 is configured to facilitate communication between the device 700 and other devices by wired or wireless means. The device 700 may have access to a wireless network based on a communication standard, such as wifi, 2G or 3G, or a combination thereof. In an exemplary embodiment, the communication component 716 receives broadcast signals or broadcast-related information from an external broadcast management system via a broadcast channel. In an exemplary embodiment, the communication component 716 further includes a near field communication (NFC) module to facilitate short range communication. For example, in the NFC module may be implemented based on Radio Frequency Identification (RFID) technology, Infrared Data Association (IrDA) technology, Ultra Wide Band (UWB) technology, Bluetooth (BT) technology and other technologies.

In exemplary embodiments, the device 700 may be implemented by one or more application-specific integrated circuits (ASICs), digital signal processors (DSPs), digital signal processing devices (DSPDs), programmable logic devices (PLDs), field-programmable gate arrays (FPGAs), controllers, microcontrollers, microprocessors, or other electronic components for performing the methods described above.

In exemplary embodiments, there is also provided a non-transitory computer-readable storage medium including instructions, such as a memory 704 including instructions, the instructions being executable by a processor 720 of the device 700 to accomplish the method. For example, the non-transitory computer-readable storage medium may be ROM, random access memory (RAM), CD-ROM, magnetic tape, floppy disks, and optical data storage devices, among others.

A non-transitory computer-readable storage medium that, when instructions in the storage medium are executed by a processor of a terminal device, enables the terminal device to perform a method of displaying a medical device of said terminal device.

The invention also provides a computer program product comprising a computer program; when executed by a processor, the computer program product implements a method as described in this embodiment.

Various embodiments of the systems and techniques described above in this disclosure can be implemented in digital electronic circuit systems, integrated circuit systems, field programmable gate arrays (FPGAs), application-specific integrated circuits (ASICs), application-specific standard products (ASSPs), systems of systems-on-a-chip (SOCs), load-programmable logic devices (CPLDs), computer hardware, firmware, software, and/or combinations thereof. These various implementations may include: implementing one or more computer programs, the one or more computer programs being executed and/or interpreted on a programmable system comprising at least one programmable processor; the programmable processor, which may be a dedicated or general-purpose programmable processor, may receive data and instructions from the storage system, the at least one input device, and the at least one output device, and transmit the data and instructions to the storage system, the at least one input device, and the at least one output device.

Program code for implementing the methods of the invention may be written in any combination of one or more programming languages. Such program code may be provided to a processor or controller of a general-purpose computer, a special-purpose computer, or other programmable data processing device, such that the program code causes the functions/operations specified in the flowchart and/or block diagram to be performed when executed by the processor or controller. The program code may be executed entirely on the machine, partially on the machine, partially on the machine as a stand-alone software package and partially on a remote machine or entirely on a remote machine or electronic device.

In the context of the invention, a machine-readable medium may be a tangible medium that may contain or store a program for use by or in conjunction with an instruction execution system, device, or apparatus. The machine-readable medium may be a machine-readable signal medium or a machine-readable storage medium. The machine-readable medium may include, but is not limited to, an electronic, magnetic, optical, electromagnetic, infrared, or semiconductor system, device, or apparatus, or any suitable combination of the foregoing. More specific examples of machine-readable storage media would include electrical connections based on one or more wires, portable computer disks, hard disks, random access memory (RAM), read-only memory (ROM), erasable programmable read-only memory (EPROM or flash memory), optical fibers, convenient compact disk read-only memory (CD-ROM), optical storage devices, magnetic storage devices, or any suitable combination of the foregoing.

To provide interaction with a user, the systems and techniques described herein may be implemented on a computer having: a display device (e.g., a cathode ray tube (CRT) or a liquid crystal display (LCD) monitor) for displaying information to the user; and a keyboard and a pointing device (e.g., a mouse or a trackball) by which the user may provide input to the computer. Other types of devices may also be used to provide interaction with the user; for example, the feedback provided to the user may be any form of sensory feedback (e.g., visual feedback, auditory feedback, or tactile feedback); and the input from the user may be received in any form, including acoustic, voice, or tactile input.

The systems and techniques described herein may be implemented in a computing system that includes back-end components (e.g., as a data electronic device), or a computing system that includes middleware components (e.g., an application electronic device), or a computing system that includes front-end components (e.g., a user's computer that has a graphical user interface or a web browser through which a user can interact with the system and techniques described herein), or any combination of such back-end components, middleware components, or front-end components. The components of the system may be interconnected by digital data communications (e.g., a communications network) in any form or medium. Examples of communication networks include: local area networks (LANs), wide area networks (WANs), and the Internet.

A computer system may include a client and an electronic device. Clients and electronic devices are generally remote from each other and typically interact over a communication network. The client-electronic device relationship is generated by a computer program that runs on a corresponding computer and has a client-electronic device relationship with each other. An electronic device may be a cloud electronic device, also known as a cloud computing electronic device or a cloud host, which is a host product in a cloud computing service system to address the shortcomings of traditional physical hosts and VPS services (“Virtual Private Server,” or “VPS” for short), which are difficult to manage and weak in terms of business scalability. Electronic devices can also be electronic devices for distributed systems, or electronic devices that incorporate block chain. It should be understood that steps can be reordered, added, or removed using various forms of processes shown above. For example, the steps documented in this application may be performed in parallel or sequentially or in a different order, as long as they achieve the desired results of the technical solutions disclosed in this application, and are not limited herein.

Other embodiments of this disclosure will readily come to mind to those skilled in the art upon consideration of the specification and practice of the invention disclosed herein. This disclosure is intended to cover any variations, uses, or adaptations of the invention that follow the general principles of this invention and include means of common knowledge or customary skill in the art not disclosed herein. The specification and embodiments are to be regarded as exemplary only, and the true scope and spirit of this invention is indicated by the following claims.

It should be understood that the invention is not limited to the precise structure which has been described above and illustrated in the accompanying drawings, and that various modifications and changes may be made without departing from its scope. The scope of this disclosure is limited only by the appended claims.

1. A method for controlling the display of a medical device, comprising:
detecting the presence of a target user within a predetermined distance of the medical device;
measuring distance between the target user and the medical device;
automatically adjusting content displayed on a display of the medical device according to the measured distance between the target user and the medical device.

2. The method according to claim 1, comprising measuring the distance between the target user and the medical device using at least one of the group of methods comprising sensing by millimeter wave radar, an infrared sensor, an ultrasonic sensor and an image acquisition device.

3. The method according to claim 2, in which the step of detecting the target user comprises determining a dwell time of a candidate user within a predetermined distance of the medical device and designating the candidate user as the target user when the dwell time exceeds a predetermined time threshold.

4. The method according to claim 2, in which the step of detecting the target user comprises determining a change of position of a candidate user within a predetermined distance of the medical device and designating the candidate user as the target user when the distance between the candidate user and the medical device is decreasing, indicating that the candidate user is moving toward the medical device.

5. The method according to claim 2, in which the step of detecting the target user comprises determining a change of position of a candidate user within a predetermined distance of the medical device and designating the candidate user as the target user when the distance between the candidate user and the medical device is decreasing, indicating that the candidate user is moving toward the medical device.

6. The method according to claim 2, further comprising:
measuring, relative to the medical device, an orientation angle of a candidate user located within a predetermined distance of the medical device;
and designating the candidate user as the target user when the orientation angle of the candidate user satisfies a preset angular orientation condition.

7. The method according to claim 2, further comprising:
acquiring a facial image of a candidate user within a predetermined distance of the medical device; and
designating the candidate user as the target user when the facial image corresponds to a prestored image that is stored in a facial image library.

8. The method according to claim 1, in which the step of automatically adjusting content displayed on a display of the medical device includes automatically changing an amount of information displayed on the display corresponding to the measured distance between the target user and the medical device, by displaying more information as the measured distance decreases and less information as the measured distance increases.

9. The method according to claim 1, in which the step of automatically adjusting content displayed on a display of the medical device includes automatically changing at least one font size of information displayed on the display corresponding to the measured distance between the target user and the medical device, by decreasing the at least one font size as the measured distance decreases and increasing the at least one font size as the measured distance increases.

10. The method according to claim 1, in which the medical device is one of a plurality of medical devices, the method further comprising:
determining which one of the plurality of medical devices is a primary medical device for the target user; and
adjusting the display of not only the primary medical device but also of at least one other of the plurality of medical devices according to the measured distance between the target user and the primary medical device.

11. The method according to claim 1, further comprising determining which of a plurality of candidate users located within the predetermined distance of the medical device is the target user.

12. An electronic device, comprising a processor and a memory in communication with the processor, wherein
the memory stores computer-executable instructions; and
the processor is configured to execute the computer-executable instructions to:
detect the presence of a target user within a predetermined distance of the medical device;
measure distance between the target user and the medical device; and
automatically adjust content displayed on a display of the medical device according to the measured distance between the target user and the medical device.

13. The electronic device of claim 12, further comprising a distance sensing device chosen from the group of millimeter wave radar, an infrared sensor, an ultrasonic sensor and an image acquisition device.

14. The electronic device of claim 12, wherein the processor is further configured to:
determine a dwell time of a candidate user within a predetermined distance of the medical device; and
designate the candidate user as the target user when the dwell time exceeds a predetermined time threshold.

15. The electronic device of claim 12, wherein the processor is further configured to determine a change of position of a candidate user within a predetermined distance of the medical device and designating the candidate user as the target user when the distance between the candidate user and the medical device is decreasing, indicating that the candidate user is moving toward the medical device.

16. The electronic device of claim 12, wherein the processor is further configured to
measure, relative to the medical device, an orientation angle of a candidate user located within a predetermined distance of the medical device; and
designate the candidate user as the target user when the orientation angle of the candidate user satisfies a preset angular orientation condition.

17. The electronic device of claim 12, wherein the processor is further configured to
acquire a facial image of a candidate user within a predetermined distance of the medical device; and
designate the candidate user as the target user when the facial image corresponds to a prestored image that is stored in a facial image library.

18. The electronic device of claim 12, wherein the processor is further configured to automatically adjust content displayed on a display of the medical device by automatically changing an amount of information displayed on the display corresponding to the measured distance between the target user and the medical device, by causing the display to display more information as the measured distance decreases and less information as the measured distance increases.

19. The electronic device of claim 12, wherein the processor is further configured to automatically adjust content displayed on a display of the medical device by automatically changing at least one font size of information displayed on the display corresponding to the measured distance between the target user and the medical device, by causing the display to decrease the at least one font size as the measured distance decreases and increasing the at least one font size as the measured distance increases.

20. The electronic device of claim 12, in which the medical device is one of a plurality of medical devices, each said device having a corresponding processor, each processor being further configured:
to determine which one of the plurality of medical devices is a primary medical device for the target user; and
to adjust the display of not only the primary medical device but also of at least one other of the plurality of medical devices according to the measured distance between the target user and the primary medical device.
